(19) United States
(12) Patent Application Publication
(10) Pub. No.: US 2003/0096408 A1
(43) Pub. Date: May 22, 2003

(54) Cell substrates and methods of use thereof

(76) Inventors: David A. Gerber, Chapel Hill, NC (US); Jian Wang, Chapel Hill, NC (US)

Correspondence Address: MYERS BIGEL SIBLEY & SAJOVEC, PO BOX 37428, RALEIGH, NC, 27627, US

(21) Appl. No.: 10/293,454

(22) Filed: Nov. 13, 2002

Related U.S. Application Data
(60) Provisional application No. 60/332,167, filed on Nov. 16, 2001.

(51) Int. Cl. C12N 5/08

(52) U.S. Cl. 435/370; 435/366

(57) ABSTRACT

A cell support system useful for the implantation of living cells in a subject comprises a solid substrate, typically formed from a biologically inert material. The substrate has a textured surface portion, with the textured surface portion defining a plurality of recessed cavities therein. A plurality of live cells to be implanted are deposited on the textured surface portion so that the cells (or progeny thereof) are protected from mechanical dislodgment.

### RELATED APPLICATIONS

This application claims the benefit of U.S. provisional application serial No. 60/332,167, filed Nov. 16, 2001, the disclosure of which is to be incorporated by reference herein in its entirety.

### FIELD OF THE INVENTION

The present invention concerns substrates, particularly microstamped or microtextured substrates, for cells such as pancreatic or hepatic cells, along with methods of using such substrates for implanting such cells in a subject.

### BACKGROUND OF THE INVENTION

One of the specialized functions of the liver includes its unique regenerative capacity, a capacity that at a cellular level varies dependent on the extent of ploidy. The liver in all adult mammals contains predominantly polyploid cells, with young adult mouse livers being ˜50% tetraploid, 40-45% octaploid and only 5-10% diploid (Weglarz, T. C. et al., American Journal of Pathology 157, 1963-1974 (2000)). The extent of polyploidy increases with age.

Two distinct forms of liver regeneration have been described since the 1930s: (1) after partial hepatectomy, the remaining tissue contains cells that undergo DNA synthesis with limited amounts of cytokinesis and results in dramatic but transient increases in tetraploid, octaploid and higher ploidy level cells and with transient reductions in the numbers of diploid cells; (2) toxic injuries (viral, chemical radiation) selectively kill the polyploid cells of the liver resulting in a cellular vacuum within each liver acinus followed by a dramatic expansion of diploid cells and secondary maturation to tetraploid and octaploid cells.

Given the known heterogeneity in phenotype among liver cells, only one component of it being the variations in ploidy, investigators have focused on whether there are specific cell populations in the liver responsible for liver regeneration. Rhim and associates evaluated the potential for replication by adult mouse hepatocytes by injecting unfractionated liver cell suspensions into transgenic mice in which the transgene consisted of an albumin promoter coupled to a construct encoding urokinase and resulting in toxicity to all cells expressing albumin. The transgene is lethal in newborns unless inoculated with liver cells. Based on the number of liver cells required to reconstitute the livers of the transgenic mice, the investigators hypothesized that adult hepatocytes could divide at least 12 times (Rhim, J. A. et al., Science 263, 1149-1152 (1994)). In parallel studies, Overturf et al. made use of mutant mice with a tyrosinemia syndrome that created a cellular vacuum within the mouse livers (Overturf, K. et al., American Journal of Pathology 151, 1273-1280 (1997)). Unfractionated adult liver cells with specific markers were injected into the mutant mice, and then the livers of the mutant mice were serially transplanted and tested for the marked cells to test the potential of adult liver cells to divide. It was claimed that the mature liver cells are capable of a series of doublings equivalent to a 7.3×10²⁰ fold expansion of the original population by serial transplantation procedures (Id.). Many liver repopulating studies have focused on the proliferation of adult mature hepatocytes as this population typically accounts for most of the replacement of host liver tissue (Sell, S., Hepatology 33, 738-750 (2001)).

When mature parenchymal cell proliferation is inhibited or when mature parenchyma are eliminated by toxic insults, a cellular vacuum is created in which the remaining cellular subpopulations, assumed to be diploid ones, are capable of extensive growth or liver reconstitution ability (Rhim, J. A. et al., Proceedings of the National Academy of Sciences of the United States of America 92, 4942-6 (1995); Sandgren, E. P. et al., Cell 66, 245-56 (1991); Overturf, K. et al., Human Gene Therapy 9, 295-304 (1998)). This has supported the increasing interest in whether progenitor cells, including stem cells, exist within adult liver tissue and whether they are merely the targets for pathogenic processes or whether they are the ultimate source for liver turnover in both normal and disease processes (Grisham, J. W. & Thorgeirsson, S. S. Liver stem cells. in Stem Cells (ed. Potter, C. S.) 233-282 (Academic Press, London, 1997); Fausto, N., Journal of Hepatology 32, 19-31 (2000); Brill, S. et al. Proceedings of the Society for Experimental Biology & Medicine 204, 261-9 (1993); Reid, L. M., Molecular Biology Reports 23, 21-33 (1996); Reid, L. M. Stem Cell/Lineage Biology and Lineage-Dependent Extracellular Matrix Chemistry: Keys to Tissue Engineering of Quiescent Tissues such as Liver, in Principles of Tissue Engineering (eds. Lanza, R., Langer, R. & Chick, W.) 481-514 (R.G. Landes Company, 1997); Sigal, S. H. et al., American Journal of Physiology 263, G139-48 (1992); Dabeva, M. D. et al., American Journal of Pathology 156, 2017-2031 (2000); Gupta, S. et al., American Journal Physiological Gastrointestinal Liver Physiological 279, G815-G826 (2000)).

In vivo, hepatocytes divide once or twice and return to quiescence after 70% hepatectomy. The mitotically dormant state of the hepatocytes of the adult liver is supported by a rate of turnover of normal liver cells that has been estimated to be 1 in 20,000 to 40,000 cells at any given time. Therefore it has been estimated that normal liver is replaced by routine tissue renewal approximately once a year (Sell, S., Hepatology 33, 738-750 (2001)). Placed in culture, hepatocytes do not undergo DNA replication unless growth factors are added to the medium. Even then, replication of hepatocytes in primary culture maintained under conventional conditions is limited (Fausto, N., Journal of Hepatology 32, 19-31 (2000)).

S. Sell recently outlined the tremendous regenerative capacity of the liver into 3 levels of cells that can respond to the loss of hepatocytes. This includes the mature hepatocyte, which responds to partial hepatectomy, centrolobular injury, and certain models of hepatocarcinogenesis; the ductal “bipolar” progenitor cell, which responds to centrolobular injury when the proliferation of hepatocytes is inhibited, and to hepatocarcinogenesis models that inhibit mature hepatocyte proliferation; and the periductular stem cell, which may be derived from circulating hematopoietic stem cells, and responds to periportal injury or to select models of hepatocarcinogenesis (Sell, S., Hepatology 33, 738-750 (2001)).

The lack of a sufficient number of donor livers to meet the transplantation needs of patients with liver diseases creates a need for novel approaches to implant liver cells. A similar demand is present for pancreatic cell transplantation. In addition, there is a need for systems which permit the ready implantation and subsequent withdrawal of cells, such as may be desired in pharmacological studies involving candidate drugs.

### SUMMARY OF THE INVENTION

A first aspect of the present invention is, accordingly, a cell support system useful for the implantation of living cells in a subject. The support comprises a solid substrate, typically formed from a biologically inert material (e.g., an organic or inorganic material). For implantation in a subject the support is preferably sterile, except for the specific cells deposited thereon for implantation as described below. The substrate preferably has a textured surface portion, with the textured surface portion defining a plurality of recessed cavities therein. The cavities may be in any form, including random or patterned pits, channels, cavities, holes, grooves, etc. A plurality of live cells to be implanted (e.g., stem or progenitor cells) are deposited on the textured surface portion, preferably in the recessed cavities (although some may be outside the recessed cavities, or the cells may be allowed to proliferate into the recessed cavities) so that the cells (and/or progeny thereof) are protected from mechanical dislodgment therefrom, in which case the cells might otherwise migrate to undesired locations within the subject and cause pathological conditions such as emboli.

Thus in a preferred embodiment of the invention the cells deposited on the substrate are not encapsulated or further coated, and are free of any overlying layers or materials, so that the implanted cells are in direct contact with the tissue of the host subjects into which they are subsequently implanted as described below. In another embodiment the cells deposited may be further encapsulated with an overlying semipermeable encapsulating layer or membrane, as discussed in greater detail below.

A second aspect of the present invention is a method of implanting cells in a subject, comprising the steps of: (a) providing a cell support as described above, and then (b) implanting the cell support in the subject.

Supports and methods as described above may be used for any suitable purpose, including but not limited to treating subjects afflicted with diabetes. In this case the cells to be implanted are pancreatic cells, and the pancreatic cells are implanted in the subject in an amount sufficient or effective to treat diabetes (in general, from about 10³ to 10⁵ cells).

The foregoing and other objects and aspects of this invention are explained in greater detail in the drawings herein and the specification set forth below.

### BRIEF DESCRIPTION OF THE DRAWINGS

FIGS. 1a-f. Colony formation from a hepatic progenitor cell after culture in standard culture medium with the addition of 1% DMSO on day 4. The cells are evident at day 1 (a). By day 4 a small colony is seen (b) and this continues to expand from day 5 (c), day 6 (d), day 7 (e) and day 8 (f). The colonies continue to grow through the first 21 days of culture. 10× magnification.

FIGS. 2a-l. Hepatic progenitor cell colonies were isolated at days 7, 14, 21 and 40 (2C, 2R, 2I, 2L) and stained in green for expression of A6, an oval cell marker (2A, 2D, 2G, 2J) or they were stained in red for albumin (2B, 2E, 2H, 2K).

FIG. 3. The percentage of cells that expressed either A6 or albumin during primary culture conditions is established. This graph represents an average of 3 independent experiments.

FIG. 4. A hepatic chip demonstrating several hepatic progenitor cells in culture at day 2 (10× magnification).

FIG. 5. Demonstrates a hepatic chip with 2 hepatic progenitor cells each located within an individual well at day 2 of culture (40× magnification).

FIG. 6 shows murine islet cells (pancreatic progenitor cells) established in culture, demonstrating colony formation and cellular expansion at day 5 (6A), day 14 (6B), and day 28 (6C).

FIG. 7 shows a pancreatic progenitor cell colony as described in FIG. 6 at day 42 of culture. The top image demonstrates a transmission image of the cell colony, while the bottom image shows cells stained with BrdU to demonstrate proliferation.

FIG. 8 shows islet/pancreatic progenitor cells at day 7 (A, B), day 14 (C, D) and day 28 (E, F) stained with A6 (red) and nestin (green). The cells expressing A6 are seen throughout the colony of islet progenitor cells while the nestin positive cells are only seen around the periphery of the islet progenitor cell colony.

### DETAILED DESCRIPTION OF THE PREFERRED EMBODIMENTS

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Applicants specifically intend that all patent references cited herein be incorporated herein by reference in their entirety.

A. Definitions

As used herein, a mammal refers to human and non-human primates and other mammals including but not limited to human, mouse, rat, sheep, monkey, goat, rabbit, hamster, horse, cow pig, cat, dog, etc.

“Non-human mammal”, as used herein, refers to any mammal that is not a human; '7non-human primate” as used herein refers to any primate that is not a human.

“Allogeneic” refers to genetically different members of the same species.

“Isogeneic” refers to of an identical genetic constitution.

“Xenogeneic” refers to members of a different species.

An “immunosuppressive agent” is any agent that prevents, delays the occurrence of or reduces the intensity of an immune reaction against a foreign cell in a host, particularly a transplanted cell.

“Stem cell” as used herein refers to an undifferentiated cell which is capable of essentially unlimited propagation either in vivo or ex vivo and capable of differentiation to other cell types. This can be differentiation to certain differentiated, committed, immature, progenitor, or mature cell types present in the tissue from which it was isolated, or dramatically differentiated cell types. In general, stem cells used to carry out the present invention are progenitor cells, and are not embryonic, or are “nonembryonic”, stem cells (i.e., are not isolated from embryo tissue). Stem cells can be “totipotent,” meaning that they can give rise to all the cells of an organism as for germ cells. Stem cells can also be “pluripotent,” meaning that they can give rise to many different cell types, but not all the cells of an organism. Stem cells can be highly motile. Stem cells are preferably of mammalian or primate origin and may be human or non-human in origin consistent with the description of animals and mammals as given above. The stem cells may be of the same or different species of origin as the subject into which the stem cells are implanted.

“Progenitor cell” as used herein refers to an undifferentiated cell that is capable of substantially or essentially unlimited propagation either in vivo or ex vivo and capable of differentiation to other cell types. Progenitor cells are different from stem cells in that progenitor cells are viewed as a cell population that is differentiated in comparison to stem cells and progenitor cells are partially committed to the types of cells or tissues which can arise therefrom. Thus progenitor cells are generally not totipotent as stem cells may be. As with stem cells, progenitor cells used to carry out the present invention are preferably nonembryonic progenitor cells. Progenitor cells are preferably of mammalian or primate origin and may be human or non-human in origin consistent with the description of animals and mammals as given above. The progenitor cells may be of the same or different species of origin as the subject into which the progenitor cells are implanted.

“Essentially unlimited propagation” can be determined, for example, by the ability of an isolated stem cell to be propagated through at least 50, preferably 100, and even up to 200 or more cell divisions in a cell culture system.

A “pancreatic” stem or progenitor cell means a stem or progenitor cell that has been isolated from pancreatic tissue and/or a cell that has all of the characteristics of: nestin-positive staining, nestin gene expression, cytokeratin-19 negative staining, long-term proliferation in culture, and the ability to differentiate into pseudo-islets in culture.

A “liver” stem or progenitor cell means a stem or progenitor cell that has been isolated from liver tissue and/or a cell that has all of the characteristics of: nestin-positive staining, nestin gene expression, and long-term proliferation in culture.

B. Solid Supports

Solid supports used to carry out the present invention can take any of a variety of forms. In general, the solid supports are formed from a stable or inert material (i.e., a material that does not substantially erode or degrade after implantation) as opposed to a biodegradable or bioerodable material. The solid support may be comprised of, consist of, or consist essentially of a polymeric or non-polymeric material, and may be comprised of, consist of, or consist essentially of an organic or inorganic material. For example, in one preferred embodiment, the solid support is formed of a semiconductor or microelectronic material that comprises, consists of, or consists essentially of one or more layers such as silicon dioxide, silicon nitride, polysiloxane and/or metal, etc.). Aluminum-aluminum oxide, gallium arsenide, ceramic, quartz or copper substrates also may be employed, as well as glass substrates, indium tin oxide (ITO) coated substrates and the like.

An electrode, sensor or the like may be fabricated on or into the substrate in accordance with known techniques, to monitor either the exogenous cells on the device, or other compounds in the subject, such as glucose.

The solid substrate may take any suitable form, such as flat, spherical, round, pyramidal, conical, irregular, etc. In one embodiment the solid substrate is substantially flat and planar, taking the appearance of a “chip” or “microchip”. In such a case, the surface portion for cell deposition may be formed on a single side, or both sides, of the substrate. In general, the substrate may range from those having a surface portion for cell deposition of from about one-half or one square centimeter up to about 25 or 30 square centimeters. Preferably the substrate is of a size sufficient to permit handling and manipulation by a surgeon during implantation, either manually or with the aid of a surgical device.

As noted above, the substrate has a textured surface portion upon which cells to be implanted are deposited. Any form of texturing may be employed, including grooves, wells, ridges, random or patterned features, etc. In general, the texturing will form raised regions or portions and lowered regions or portions on the overall surface portion such that cells carried by the surface portion may reside or be positioned, in whole or in part, in the lowered portions, with the raised portions protecting the cells from mechanical dislodgement during manipulation or handling of the substrate. The vertical distance from raised portion to lowered portion will vary in particular devices depending upon the cells to be implanted, but in general will be at least as much as about one-half the vertical height of a cell deposited in the lowered portion (i.e. the vertical distance may be at least about 1 micron, up to about 10, 20 or 50 microns or more:).

Texturing of the surface portion may be achieved through any suitable fabrication technique, including but not limited to microstamping, lithography, etching, casting, dissolution, etc.

Deposition of the stem cells on the surface may be carried out by any suitable means which is not unduly toxic or disruptive of the living cells being deposited. In a preferred embodiment, cells may be deposited by a micropipette or other micromanipulator in a sterile aqueous solution, with cells depositing on the surface by gravity and adhering to the surface portion by virtue of cell surface proteins that have adhesive properties with respect to the surface portion. Optionally, the chip may be coated with an extracellular matrix such as collagen, laminin, or the like to promote adhesion of cells. After cell deposition, the chip or substrate is preferably maintained in a sterile aqueous oxygenated solution such as a nutrient solution until the substrate with the cells is implanted in the host or subject as discussed below.

C. Cells

Progenitor and stem cells used to carry out the present invention may be obtained and produced by any suitable procedure, including the procedures described herein and procedures known in the art. In general, the cells of the invention are not embryonic stem cells, but are rather nonembryonic stem or progenitor cells that give rise to a particular type or category of progeny cells (e.g. liver progenitor or stem cells used in the invention may give rise to hepatocytes and biliary cells; pancreatic stem or progenitor cells used in the invention may give rise to acinar cells, islet cells, and/or ductal cells, etc.). Thus examples of progenitor or stem cells that may be used to carry out the present invention include liver cells, pancreatic cells, intestinal cells, renal cells, and epithelial cells.

Examples of suitable cells for carrying out the present invention, and/or manners of isolating the same, include but are not limited to those described in: Zulewski, H., et al., Multipotential nestin-positive stem cells isolated from adult pancreatic islets differentiate ex vivo. Diabetes, 2001. 50(5): p. 521-533. Clatworthy, J. P. and V. Subramanian, Stem cells and the regulation of proliferation, differentiation and patterning in the intestinal epithelium: emerging insights from gene expression patterns, transgenic and gene ablation studies. Mechanisms of Development, 2001. 101(1-2): p. 3-9.; Alessandri, G., et al., Human vasculogenesis ex vivo: embryonal aorta as a tool for isolation of endothelial cell progenitors. Laboratory Investigation, 2001. 81(6): p. 875-85; Shintani, S., et al., Mobilization of enclothelial progenitor cells in patients with acute myocardial infarction. Circulation, 2001. 103(23): p. 2776-9; Bonner-Weir, S., et al., In vitro cultivation of human islets from expanded ductal tissue. Proc. Natl. Acad. Sci. USA 97(14): p. 7999-8004; Suzuki, A., et al., Flow-cytometric separation and enrichment of hepatic progenitor cells in the developing mouse liver, Hepatology, 2000. 32(6): p. 1230-1239; Lagasse, E., et al., Purified hematopoietic stem cells can differentiate into hepatocytes in vivo. Nature Medicine, 2000. 6(11): p. 1229-1234; Alison, M. R., et al., Hepatocytes from non-hepatic adult stem cells. Nature, 2000. 406: p. 257; Ramiya, V. K., et al., Reversal of insulin-dependent diabetes using islets generated in vitro from pancreatic stem cells. Nature Medicine, 2000. 6(3): p. 278-282; U.S. Pat. No. 6,436,704 to Roberts et al. (Human pancreatic epithelial progenitor cells and methods of isolation and use thereof); U.S. Pat. Nos. 6,365,385 and 6,303,355 to Opara (Method for culturing, cryopreserving, and encapsulating pancreatic islet cells); U.S. Pat. No. 6,326,201 to Fung et al., (Pancreatic progenitor cells, methods and uses related thereto); U.S. Pat. No. 6,129,911 to Faris, (Liver Stem Cell); U.S. Pat. Nos. 6,399,341 and 6,023,009 to Stegemann et al. (Artificial Pancreas); U.S. Pat. No. 6,197,575 to Griffith et al., (Vascularized perfused microtissue/micro-organ arrays); U.S. Pat. No. 6,001,647 to Peck et al. (In vitro growth of functional islets of Langerhans and in vivo uses thereof); U.S. Pat. No. 5,919,703 to Mullen et al., (Preparation and storage of pancreatic islets); U.S. Pat. No. 5,888,705, Rubin et al.; (Compositions and method of stimulating the proliferation and differentiation of human fetal and adult pancreatic cells ex vivo); U.S. Pat. No. 5,855,616 to Fournier et al. (Bioartificial pancreas); U.S. Pat. No. 5,821,235, to Henning et al. (Gene therapy using the intestine), U.S. Pat. No. 5,786,340 to Henning et al. (Gene transfer to the intestine), U.S. Pat. No. 5,681,587, Halberstadt et al. (Growth of adult pancreatic islet cells); U.S. Pat. No. 5,587,309, Rubin et al. (Method of stimulating proliferation and differentiation of human fetal pancreatic cells ex vivo); and U.S. Pat. No. 5,559,022 to Naughton et al. (Liver reserve cells).

In one preferred embodiment, the cells are left uncoated or unencapsulated on the substrate after deposition. However, as noted below, encapsulation may be desired in some embodiments, such as to reduce graft vs. host disease or rejection. In such cases, any suitable material may be used to encapsulate the cells (i.e., provide an encapsulating layer over the cells), so long as the encapsulating layer is a “semipermeable” layer: that is, impermeable to the implanted cells and host cells, particularly host immune cells, permeable to nutrients or other materials (e.g., toxins in the case of liver cells) to be carried into the cells and permeable to waste or other materials (e.g., insulin in the case of pancreatic cells) to be secreted or excreted by the cells. Any suitable coating may be employed as an encapsulating layer, including but not limited to alginate as described in U.S. Pat. No. 6,365,385 to Opara et al. Since such semipermeable encapsulating layers may be quite fragile, the solid support still advantageously lends structural support and stability to the cells to be implanted.

D. Implantation Procedures

Progenitor or stem cells may be isogeneic, allogeneic or even xenogeneic with respect to the host or subject into which they are implanted. In general, it is preferred that the cells be mammalian and the subject be mammalian, and in one embodiment both the cells and the subject are human. When allogeneic or xenogeneic transplantation or implantation of stem cells is carried out, graft versus host rejection can be treated by appropriate encapsulation of the stem cells, by immunologically blinding of the stem cells (e.g., as described in PCT Application WO 01/39784 to Abraham et al.), or by treating the subject or host with an immunosuppressive agent in accordance with known techniques. Treatment with an immunosuppressive agent can be accomplished by administering a subject any agent which prevents, delays the occurrence of or decreases the intensity of the pertinent immune response, e.g., rejection of transplanted cells. For example, a host or subject may be administered an immunosuppressive agent that inhibits or suppresses cell-mediated immune responses against cells identified by the immune system as non-self. Examples of such immunosuppressive agents include, but are not limited to, cyclosporin, cyclophosphamide, prednisone, dexamethasone, methotrexate, azathioprine, mycophenolate, thalidomide, tacrolimus, rapamycin, systemic steroids, as well as a broad range of antibodies, receptor agonists, receptor antagonists, and other such agents as known to one skilled in the art. Various other strategies and agents can be utilized for immunosuppression. For example, an antibody, such as an anti-GAD65 monoclonal antibody, or another compound which masks a surface antigen on a transplanted cell and therefore renders the cell practically invisible to the immune system of the host, can be administered to the cells being implanted prior to implantation thereof.

Cells to be implanted may be deposited on the substrate by any suitable technique, as discussed above. In general, between about 10² or 10³ up to about 10⁶ or 10⁸ cells, carried by one or more substrates, are implanted. Any number of substrates carrying the desired number of cells may be implanted, typically from 1 to 4, 6 or 10 or more. Implantation can be into any suitable tissue that provides the desired contact of the stem cells to the host, as discussed below.

For example, a substrate of the invention may be implanted in a muscle such as an abdominal or lumbar muscle, or even an extremity muscle such as a quadricep or hamstring muscle. Muscle is a useful implantation region because it is highly vascularized. For muscle implantation, a small incision may be made through the muscle fascia so that the substrate may be implanted directly into the muscle tissue itself to maximize potential vascular contact.

Other regions for implantation include the liver. This may be carried out by implantation directly into the liver parenchyma, by implantation into the portal vein or a branch of the portal vein, etc. Additional regions for implantation include the peritoneal cavity where delivery may be carried out by minimally invasive surgical approaches as include laparoscopy.

Introduction of the substrate into a tissue can be carried out by direct surgical implantation or by introduction with the assistance of a surgical aid such as a catheter-based delivery system. In a preferred embodiment, the cells carried by the substrate are not encapsulated or surface coated (as is done with other types of artificial organs) so that, once implanted, the stem cells are in direct contact with the host (host tissue, host blood, etc.).

It will also be appreciated that cells may be implanted in vivo in a subject yet be physically external to the body of the subject, such as by containing the cells within a catheter, which receives fluid such as blood from the body and then returns that blood to the body, such as by a venous to venous shunt.

Substrates of the invention may be used as artificial organs for the treatment of type 1 diabetes (i.e., where pancreatic stem cells are carried by the substrate), for genetic disorders related to the liver or inherited diseases of the liver (e.g., where liver stem or progenitor cells are carried by the substrate). Examples of suitable subjects for implantation of liver cells with the methods and products of the present invention include but are not limited to human or animal subjects afflicted with Alagille syndrome, alcoholic liver disease, alpha-1-antitrypsin deficiency, autoimmune hepatitis, Budd-Chiari syndrome, biliary atresia, Byler disease, cancer of the liver, Caroli disease, virrhosis of the liver, Crigler-Najjar syndrome, Dubin-Johnson syndrome, fatty liver, galactosemia, Gilbert syndrome, glycogen storage disease, hemangioma of the liver, hemochromatosis, hepatitis (including hepatitis A, B, C, D, E, and G), porphyria cutanea tarda, primary biliary cirrhosis, erythrohepatic protoporphyria, rotor syndrome, sclerosing cholangitis, Wilson disease, etc. It will be appreciated that, in one embodiment, cells may be implanted by the method of the present invention as an intermediate step to assist the patient's own liver while the patient's liver heals, while the patient awaits a liver transplant, etc.

In addition to treating disease and/or administering an active agent to a subject, substrates of the invention may be used to create a surrogate organ in a subject that is being administered a test compound such as a potential new pharmaceutical treatment during a clinical trial which can be conveniently removed (after proliferation and/or engraftment and functioning of the cells within the host) from the subject for examination after treatment. The clinical trial may be of any suitable type of drug or drug candidate, including but not limited to antihypertensive compounds, anticancer or antineoplastic compounds, psychoactive compounds such as antidepressant or antischizophrenic compounds, antinausea drugs, etc., which drugs may be proteins, peptides, antibodies, small organic compounds, etc.). It will be appreciated that the “clinical trial” may be in animal subjects for safety and/or efficacy purposes, whether the drug is intended for human or animal therapy. Where multiple substrates are implanted, individual substrates may be removed at different points in time to examine the response of the cells to a particular treatment over time. Cells can be examined after removal by any suitable technique, such as histology/microscopy, bioassay, etc. Any of a variety of stem or progenitor cell types may be implanted for this purpose, including liver cells, pancreatic cells, intestinal cells, renal cells, epithelial or skin cells, or any other suitable cell.

The present invention is explained in greater detail in the following non-limiting Examples.

### EXAMPLE 1

The following examples focus on the isolation and characterization of a hepatic progenitor cell population isolated from untreated adult mouse livers and subsequently demonstrates tremendous proliferative potential that is not characteristic of mature hepatocytes. Such cells are cell therapy candidates to reconstitute damaged livers. In addition, the design of an implantable renewable bioartificial liver is described as a possible alternative to intravascular transplantation of these cells.

A. Methods

Mice. C57BL/6 mice were purchased from the Jackson Laboratory (Bar Harbor, Me.). All animals were maintained on a rodent chow under a constant day/night cycle. Three- to six-week old mice were used in all experiments. All experiments were conducted in accordance with the principles and procedures outlined in the NIH Guide for the Care and Use of Laboratory Animals.

Green fluorescent mice. The “green mouse” is a transgenic mouse on a C57BL/6 background; the transgene consists of a gene encoding “enhanced green fluorescent protein” (“eGFP”) that has been injected into fertilized eggs. The eGFP was introduced into an expression vector containing the chicken β-actin promoter and a CMV enhancer and an intron from the beta-actin gene. This approach allows efficient cloning of cDNA into the vector. Excitation/emission wavelengths for eGFP are 488/522 nm (Serody, J. S. et al., Blood 96, 2973-2980 (2000)). These mice are the kind gift of Dr. Jon Serody at the Lineberger Cancer Center at the University of North Carolina.

Hepatocyte Isolation and Culture. Reagents in these experiments were from Sigma, St. Louis, Mo. unless otherwise stated. Hepatocytes were isolated using a modification of the two-stage liver perfusion technique described by Seglen (Methods Cell Biol. 13, 29-83 (1976)). The liver was perfused with KRH buffer with 100 U/ml collagenase I solution. After digestion the cell suspension was centrifuged at 45 g for 1 minute. The supernatant fraction was resuspended and centrifuged at 45 g for 2 minutes. The supernatants were pooled and centrifuged at 120 g for 5 minutes. This cell fraction was further referred to as the “S” fraction. The pellet from the initial centrifugation was subsequently referred to as the “P” fraction. Cell viability by trypan blue exclusion test demonstrated 87-95% viability in the pellet fraction (primarily mature hepatocytes) with ≧95% viability in the “S” fraction. The “S” fraction was plated on tissue culture dishes coated with collagen I in 2 ml of DMEM (Dulbecco's Modified Eagle's medium), 10% FBS, 10 mM nicotinamide, 1 mM Asc2P (ascorbic acid 2-phosphate), 10 ng/ml EGF (epidermal growth factor), ITS (insulin, transferrin, selenium), and dexamethasone. Cellular density was 8×10⁵ cells per 35-mm well. The cells were cultured in a 5% CO₂/95% room air incubator at 37° C. 1% DMSO (dimethyl sulfoxide) was added four days after the primary cultures were established.

Antibodies. Commercially prepared antibodies include anti-rat albumin antibody (ICN), mouse anti-human CK 7 (Chemicon, Temecula, Calif.), anti-mouse H-2 K^{b} (BD Pharmingen), and an anti-mouse CD54 (BD Pharmingen). The “A6” antibody (a surface-exposed component shared by mouse oval and biliary epithelial cells, raised against dipin-induced hepatocarcinogenesis) was a kind gift from V. Factor (Engelhardt, N. V. et al., Differentiation 45, 29-37 (1990); Engelhardt, N. V. et al., Differentiation 55, 19-26 (1993)). The secondary antibody used was a Texas red goat anti-rat (Molecular Probes; Eugene, Oreg.).

Immunofluorescence. Cultured cells were washed twice with DMEM and then fixed with 1% paraformaldehyde. 1% saponin was added to the dish for a final concentration of 0.05% for analysis of intracytoplasmic antigens.

Controls included staining with isotype-matched irrelevant antibodies. Slides were examined with a Zeiss Axiocarvert 100 direct view real time white light confocal phase fluorescence microscopy system with an axiocam digital camera.

Flow Cytometry. For flow cytometric analysis of cytoplasmic antigens (e.g. albumin, AFP), the cells were fixed in 3% paraformaldehyde and permeabilized prior to staining with the antisera. Cells were stained for immunofluorescence with commercially available antibodies and either fluorochrome-conjugated secondary antibodies or a β-galactosidase biotin-streptavidin of FITC and TRITC for characterization of the cells.

Confocal Microscopy. The cells were suspended in Hanks Buffered Saline Solution (HBSS) supplemented with 1% bovine serum albumin (BSA), insulin (5 μg/ml), transferrin (5 μg/ml), and selenium (10⁻⁹M). Of the available lasers, the argon-krypton laser with 488 and 568 nm lines can simultaneously excite green-fluorescing dye like fluorescein (FITC) and red-fluorescing dye like phycoerythrin (PE). Red and green fluorescence can be detected simultaneously using different photomultipliers. For confocal analysis of cytoplasmic antigens the cells were fixed with 4% paraformaldehyde and permeabilized prior to staining with the antisera. Cells were stained as indicated above for immunofluorescence but using antibodies directly labeled with the relevant fluoroprobe. Co-loading of two or more markers was performed. Multiple channel measurements of fluorescence allowed the identification of cell types in the same sample. 50,000 cells/sample were loaded on a micro slide with Cytospine3 at 1000 rpm for 5 minutes. The analysis was done with a LSM410 laser scanning confocal microscope.

Statistics and Data Analysis. Where appropriate values were given as mean±SD.

B. Results

Identification and Isolation of Hepatic Progenitors. The heterogeneous cell population of the adult liver includes mature biliary cells and mature hepatocytes as well as progenitors and non-parenchymal cells. Using modifications of previously described techniques by Seglen et al. a cellular population was isolated containing small parenchymal cells and non-parenchymal cells through a combination of mechanical and enzymatic digestion steps (Seglen, P. O., Methods Cell Biol 13, 29-83 (1976)). By performing serial centrifugations, a distinct cellular fraction, referred to as the hepatic progenitor (HP) cell was found within the supernatant or “S” fraction. The pellet (“P” fraction) that is generated from this technique contains the mature hepatocytes, 40-50μ in diameter along with numerous non-parenchymal cells (i.e. Kupffer cells, stellate cells). The “S” fraction primarily contains the hepatic progenitor cells (3-15μ diameter) and some contaminating non-parenchymal cells. Purity of the supernatant population was obtained by modifying the centrifugation method of these cells. The two cellular fractions (as determined by body weight of the animal) yield 0.8-1.2×10⁶ cells/body weight within the “S” fraction and 2.2-3.8×10⁶ cells/body weight within the “P” fraction.

Purity of the “S” fraction was enhanced through two steps. First the cells were cultured in DMEM which is felt to suppress the growth of non-parenchymal cells (Mitaka, T. et al., Journal of Gastroenterology and Hepatology 13 (Suppl.), S70-77 (1998)). Second, the addition of dimethyl sulfoxide (DMSO) at day #4 of culture is believed to prevent replication of mature hepatocytes and other mature cells. DMSO has also been shown to inhibit EGF (epidermal growth factor) and HGF (hepatocyte growth factor)-mediated DNA replication of hepatocytes in short-term culture in a dose-dependent manner (Pagan, R. et al., Journal of Hepatology 31, 895-904 (1999); Kost, D. P. & Michalopoulos, G. K., Journal of Cellular Physiology 147, 274-80 (1991)).

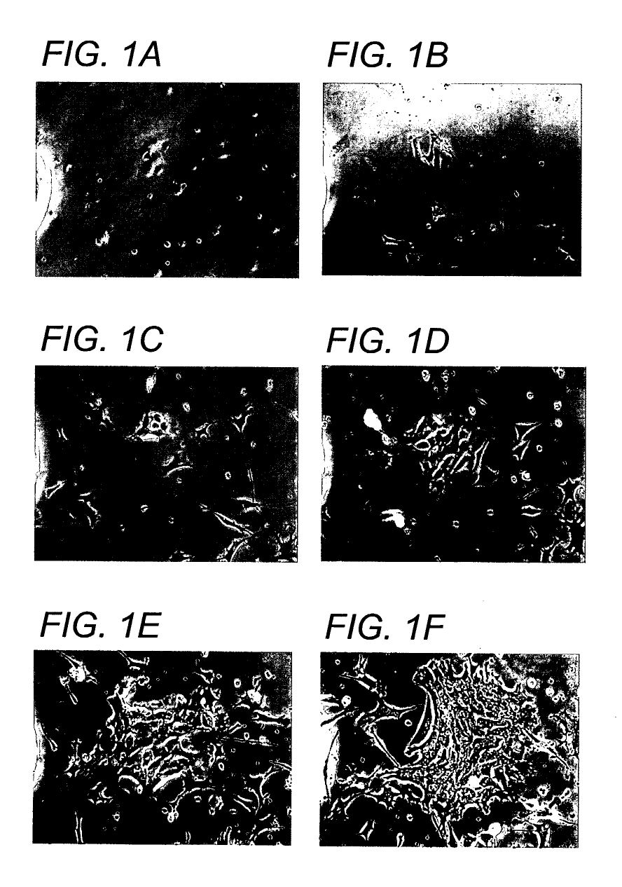

Colony formation of small hepatocytes. Maximal colony formation of the hepatic progenitors was demonstrated using in vitro conditions established in these studies. These conditions generate an average of 27 colonies/8×10⁵ cells from the “S” fraction/dish (range: 10-93 colonies/dish). The colonies are subdivided into large and small colonies, based on the number of cells per colony. A colony with >150 cells is delineated as a large colony, and those with ≦150 cells are a small colony. The distribution of colonies is roughly 50:50. Of note, most colonies can be followed from their inception at day 2 or 3 through their early proliferation beginning at days 4-5. Colony growth can be subsequently followed with colonies continuing to expand through 14 days of primary culture. [FIGS. 1a-f] The hepatic progenitor cells have been maintained in primary culture conditions for >150 days, a unique finding compared with the observations typical for mature hepatocytes that can be maintained in culture for only 4-5 days.

Sub-culture experiments. After 14 days of primary culture collagenase was used to release all of the cells and colonies from the dishes in an effort to perform sub-culture experiments. The cells were re-plated on a collagen I coated dish using the same culture conditions as the primary culture. We demonstrated a slight increase in the number of hepatic progenitor colonies by a factor of 1.03 compared to the original number of colonies on the primary dish. There was an average of 29 colonies on the secondary cultures (8-55 colonies/dish). Additional subculture experiments were established at earlier time points (between days 4-7) and they did not provide an increase in the number of colonies formed in the secondary cultures.

Additional experiments have looked at the conditions that are requisite for colony formation in the “S” fraction. Establishing these cultures with “conditioned” media from hepatic progenitor colonies in culture (early timepoints) did not enhance primary colony formation. In addition, mixed cultures were prepared by adding 10⁴ mature hepatocytes to the primary hepatic progenitor cultures (under the theory that there is one or more cellular factor(s) being produced that leads to hepatic progenitor cell growth) (Tateno, C. & Yoshizato, K., Wound Rep. Reg. 7, 36-44 (1999)). Under these conditions there are no differences in colony formation compared with primary SH cultures (Data not shown).

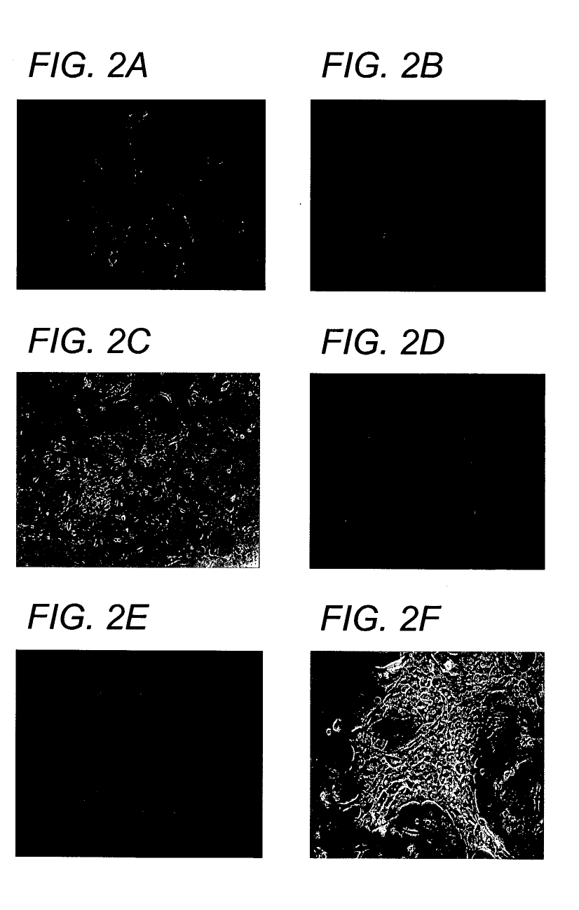

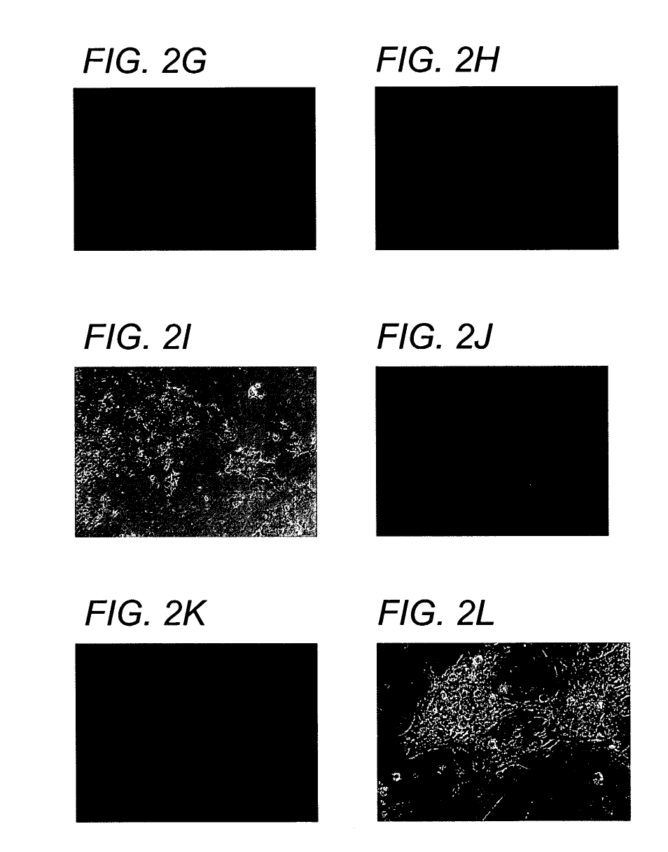

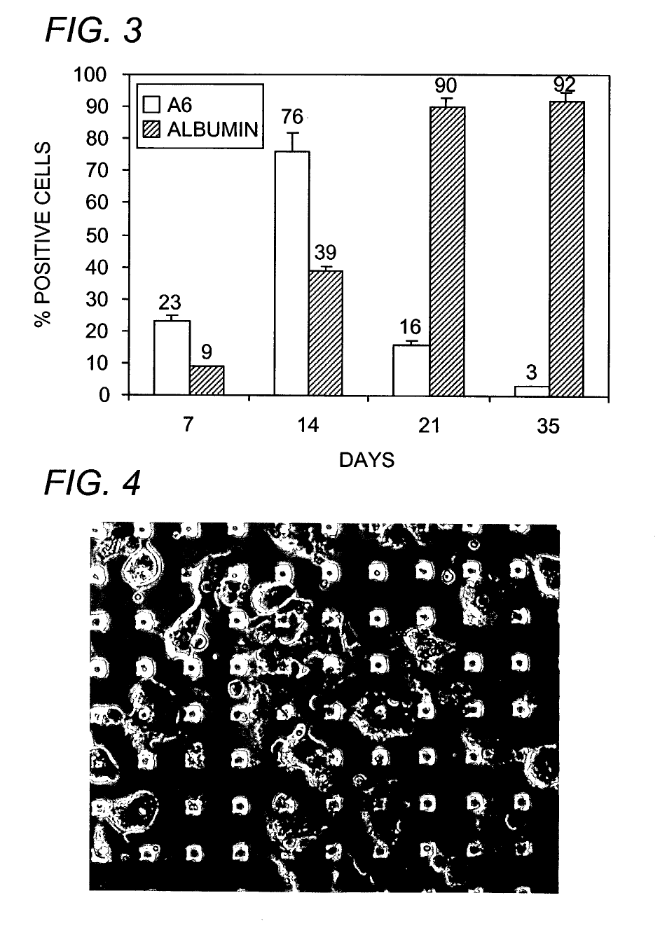

Antigenic profiles of hepatic progenitors in vitro. Further attempts at delineating differences between the hepatic progenitors and mature hepatocytes included analysis of their antigenic profiles. The hepatic progenitors do not express CD117 (c-kit), MHC Class II, a common lymphocyte marker (CD45), or markers specific for Kupffer or hepatic stellate cell populations. In addition they are not of fibroblast origin. They do demonstrate variable expression of albumin with increasing intensity from day #4 through day #21. Also, 50-55% of the cells positive for albumin are also CD54+. The greatest distinction found was that the oval cell marker, A6, is expressed beginning at days 5-7 with increasing expression through day #14 of culture and subsequently decreased expression through day 28 until there is no expression. In contrast, albumin shows increased expression on the cells from day #7 through day #28. [FIGS. 2a-l] While the albumin expression increases through day 21 of culture it appears to achieve a steady-state level of expression that persists after the first month in culture. [FIG. 3] AFP expression was also evaluated on these cells and they were initially negative for AFP expression until approximately day #28 of culture. This was analyzed by immunofluorescence after colonies began to form and the initially isolated cells on day 0 were analyzed by flow cytometry [Data not shown]. While the hepatic progenitors are shown to be ICAM-1+ throughout their period in culture, these cells are surprisingly MHC Class I negative. [Table 1] By contrast, the mature hepatocytes are positive for Class I and ICAM-1.

Hepatic Progenitor Transplantation. Hepatic progenitors were isolated from C57BL/6 mice transfected with the green fluorescent protein and cultured for 14 days until colony formation was demonstrated. 1-2×10⁵ cells in 100 μl HBSS were transplanted via tail vein infusion.

Chip design for a renewable bioartificial liver. Using a microcontact printing technique we created a geometric micropattern on a polydimethysiloxane (PDMS) stamp to produce wells that ranged from 5-200 μm in diameter on a substrate. The distance between wells ranged from 10-300 μm. The grid pattern ranged from 3-20 μm in line width and from 50-100 μm in line distance. The line widths and line distances of the pattern differ less than 0.5 μm using this PDMS stamp. The thickness of the stamps ranged 1-1.5 μm.

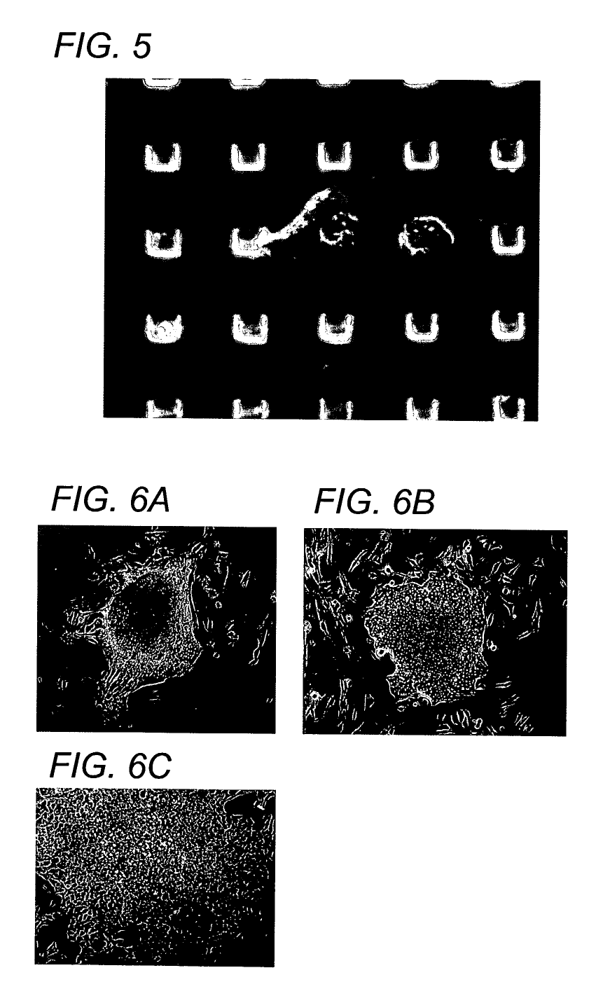

The stamps were sonicated in a freshly made 1% 3-aminopropyltriethoxysilane (Sigma, Mo.) solution in sterile distilled water for 2 minutes. After that, the stamps were rinsed with sterile distilled water and dried for 10 minutes in a 110° C. oven. Stamps were coated with collagen IV. (see, for example, Y D Kim, C B Park, D S Clark. Stable Sol-Gel Microstructured and Microfluidic Networks for Protein Patterning. Biotechnol Bioeng; vol. 73: 331-337, 2001). After 24 hours the cells were plated on the stamp at 2×10⁶ cells/ml on a 1 cm² stamp. The stamps were cultured for 1 hr in a L15 medium followed by removal of nonadherent cells. The chips were examined under a light microscope and poorly plated stamps were discarded. The cells were cultured in a 5% CO₂/95% room air incubator at 37° C. FIGS. 4 and 5.

C. Discussion

The cells that we have characterized as “hepatic progenitors” appear to be related to the “oval cells”, originally identified by Farber as immature epithelial cells with an oval shaped nucleus and scant cytoplasm that are induced to proliferate in response to treatment with chemical carcinogens in conjunction with two-thirds partial hepatectomy. The unique characteristics of the hepatic progenitor cells include their ability to be isolated and grown in vitro without previously exposing the animal to a carcinogenic insult or performing a partial hepatectomy. In the previously described in vitro system the hepatic progenitors transiently express an oval cell marker during the early stages of cellular proliferation and differentiation from days 4 through 40 with a peak expression of this marker at day 14. These cells are thought to be undergoing a state of differentiation and maturation based on the progressive increase in expression of albumin under the culture conditions. [FIG. 3] This characteristic is a distinct difference from the mature hepatocytes that make up the pellet during the initial cellular isolation from the liver. The mature hepatocytes do not express A6 at the time of isolation or in culture and they have expression of albumin, which persists but does not fluctuate as the cells remain in culture.

These hepatic progenitors have similar morphology to the small hepatocytes isolated in rat by Mitaka et al and were further characterized in experiments to determine how they compared with less well differentiated cells of hepatic origin (Mitaka, T. et al., Journal of Gastroenterology and Hepatology 13 (Suppl.), S70-77 (1998)). They also have similar characteristics to the hepatic progenitor cell and stem cell population isolated from fetal and adult rat livers (Sigal et al, 1994; Sigal et al, 1995; Brill et al, 1995) or from fetal liver sources (Kubota, H. & Reid, L. M., PNAS 97, 12132-12137 (2000); Suzuki, A. et al., Hepatology 32, 1230-1239 (2000)). The fact that these hepatic progenitor cells can be isolated from adult liver sources indicates that a modified approach to hepatocellular therapies will be more readily applied and have greater success because of the cells' proliferative potential versus transplantation with mature hepatocytes and their limited proliferative potential.

The application of cellular transplantation with distinct hepatocyte populations has been addressed in both experimental models and clinical examples. The use of mature hepatocytes has seen very little success probably due to the limited proliferation ability of the cells and their sensitivity to ischemia/reperfusion. A separate but significant challenge with hepatocellular transplantation is the transient portal hypertension and risk of pulmonary emboli associated with the infusions. These challenges led to the ongoing need for a functional bioartificial liver. Many attempts at bioartificial livers have been based on dialysis devices that are attached to a patient through a vascular catheter and subsequently used for repeat treatments. The chip design for a bioartificial liver creates a renewable organ based on a platform of cellular proliferation leading to the subsequent development of a functional organ. By patterning a chip for placement of cells we have been able to maximize the number of cells that can be placed in culture and minimized the overall size of the bioartificial organ so that it can be implanted as a therapeutic tool.

### EXAMPLE 2

This example describes the preparation of pancreatic cells useful for carrying out the present invention.

C57BL/6 mice (4-6 weeks old) are used as a source of pancreata, in accordance with known techniques. Islets are isolated by collagenase digestion of the pancreas with Collagenase V via common bile duct cannulation and the islets subsequently individually hand-picked, in accordance with known techniques. Islet pancreatic progenitor cells are then cultured in 35-mm tissue culture dishes in RPM 1640 media with 2% FBS, 12.5 mM Hepes, 11.1 mM glucose and 20 ng/ml epidermal growth factor. The media is changed every 2-3 days.

FIG. 6 shows murine islet cells (pancreatic progenitor cells) established in culture, demonstrating colony formation and cellular expansion at day 5 (6A), day 14 (6B, and day 28 (6C).

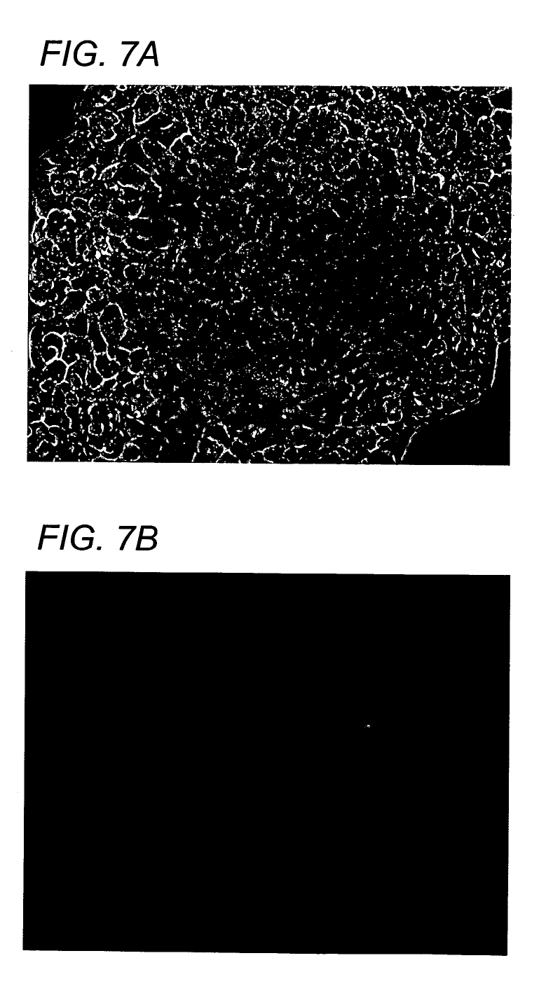

FIG. 7 shows a pancreatic progenitor cell colony as described in FIG. 6 at day 42 of culture. The top image demonstrates a transmission image of the cell colony, while the bottom image shows cells stained with BrdU to demonstrate proliferation.

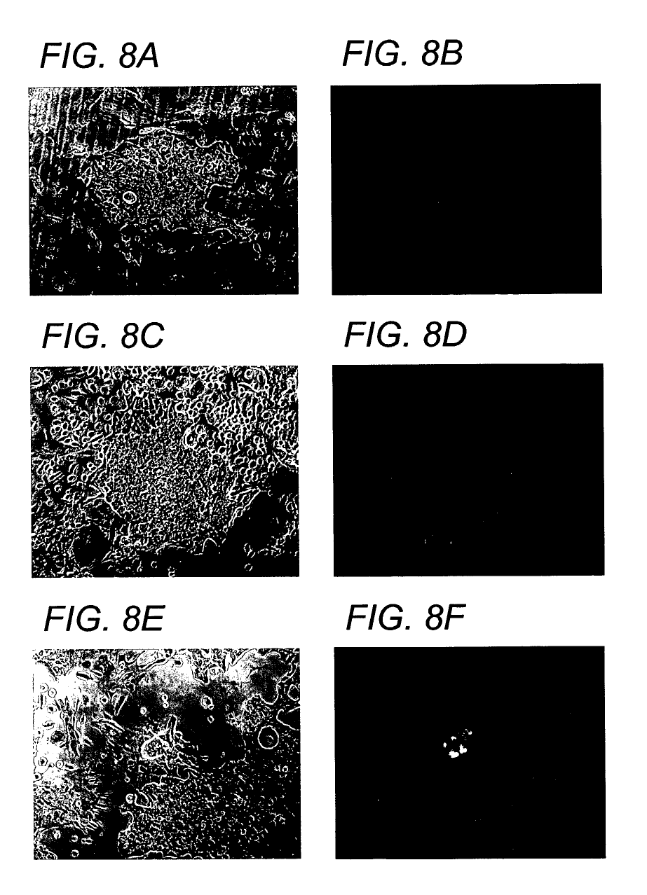

FIG. 8 shows islet/pancreatic progenitor cells at day 7 (A, B), day 14 (C, D) and day 28 (E, F) stained with A6 (red) and nestin (green). The cells expressing A6 are seen throughout the colony of islet progenitor cells while the nestin positive cells are only seen around thee periphery of the islet progenitor cell colony.

The foregoing is illustrative of the present invention, and is not to be construed as limiting thereof. The invention is defined by the following claims, with equivalents of the claims to be included therein.

That which is claimed is:

1. A cell support useful for the implantation of cells in a subject, said cell support comprising:
a solid substrate formed from a biologically inert material and having a textured surface portion, said textured surface portion defining a plurality of recessed cavities in said textured surface portion; and
a plurality of live cells deposited on said textured surface portion in said recessed cavities so that said cells are protected from mechanical dislodgment therefrom;
wherein said cells consist essentially of nonembryonic hepatic or pancreatic progenitor cells.

2. The cell support according to claim 1, wherein said substrate is substantially flat in shape.

3. The cell support according to claim 1, wherein said substrate is substantially spherical in shape.

4. The cell support according to claim 1, wherein said substrate is comprised of organic material.

5. The cell support according to claim 1, wherein said substrate is comprised of inorganic material.

6. The cell support according to claim 1, wherein said substrate is comprised of silicon.

7. The cell support according to claim 1, wherein said progenitor cells consist essentially of pancreatic progenitor cells.

8. The cell support according to claim 1, wherein said progenitor cells consist essentially of liver progenitor cells.

9. The cell support according to claim 1, wherein said textured surface portion has an area of from about on-half square centimeter up to about 30 square centimeters.

10. The cell support according to claim 1, wherein said textured surface portion is comprised of raised portions and lower portions, with the vertical distance between said raised portions and lowered portions being from about 1 micron to about 50 microns.

11. A method of implanting cells in a subject, comprising the steps of:
(a) providing a cell support comprising (i)a solid substrate formed from a biologically inert material and having a textured surface portion, said textured surface portion defining a plurality of recessed cavities in said textured surface portion, and (ii) a plurality of live cells deposited on said textured surface portion in said recessed cavities so that said cells are protected from mechanical dislodgement therefrom,
wherein said cells consist essentially of nonembryonic hepatic or pancreatic progenitor cells; and then
(b) implanting said cell support in said subject.

12. The method according to claim 11, wherein said cell support is implanted parenterally in said subject.

13. The method according to claim 11, wherein said cell support is implanted intramuscularly in said subject.

14. The method according to claim 11, wherein said cell support is implanted subcutaneously in said subject.

15. The method according to claim 11, wherein said cell support is implanted intraperitoneally in said subject.

16. The method according to claim 11, wherein said substrate is maintained in said subject for a time sufficient for said cells to proliferate on said surface portion.

17. The method according to claim 11, wherein said subject is afflicted with diabetes, wherein said progenitor cells are pancreatic progenitor cells, and wherein said pancreatic progenitor cells are implanted in said subject in an amount sufficient to treat said diabetes.

18. The method according to claim 11, wherein said substrate is substantially flat in shape.

19. The method according to claim 11, wherein said substrate is substantially spherical in shape.

20. The method according to claim 11, wherein said substrate is comprised of organic material.

21. The method according to claim 11, wherein said substrate is comprised of inorganic material.

22. The method according to claim 11, wherein said substrate is comprised of silicon.

23. The method according to claim 11, wherein said progenitor cells consist essentially of pancreatic progenitor cells.

24. The method according to claim 11, wherein said progenitor cells consist essentially of liver progenitor cells.

25. The method according to claim 11, wherein said textured surface portion has an area of from about on-half square centimeter up to about 30 square centimeters.

26. The method according to claim 11, wherein said textured surface portion is comprised of raised portions and lower portions, with the vertical distance between said raised portions and lowered portions being from about 1 micron to about 50 microns.

27. A method of making a cell support useful for the implantation of living cells in a subject, said method comprising the steps of:
(a) providing a solid substrate formed from a biologically inert material and having a textured surface portion, said textured surface portion defining a plurality of recessed cavities in said textured surface portion; and then
(b) depositing a plurality of cells on said textured surface portion in said recessed cavities so that said cells are protected from subsequent mechanical dislodgment therefrom, wherein said cells consist essentially of nonembryonic hepatic or pancreatic progenitor cells.

28. The method according to claim 27, wherein said substrate is substantially flat in shape.

29. The method according to claim 27, wherein said substrate is substantially spherical in shape.

30. The method according to claim 27, wherein said substrate is comprised of organic material.

31. The method according to claim 27, wherein said substrate is comprised of inorganic material.

32. The method according to claim 27, wherein said substrate is comprised of silicon.

33. The method according to claim 27, wherein said progenitor cells consist essentially of pancreatic progenitor cells.

34. The method according to claim 27, wherein said progenitor cells consist essentially of liver progenitor cells.

35. The method according to claim 27, wherein said textured surface portion has an area of from about one-half square centimeter up to about 30 square centimeters.

36. The method according to claim 27, wherein said textured surface portion is comprised of raised portions and lowered portions, with the vertical distance between said raised portions and lowered portions being from about 1 micron to about 50 microns.

37. A method for the implantation of cells in a subject, said method comprising the steps of:
(a) providing a solid substrate formed from a biologically inert material and having a textured surface portion, said textured surface portion defining a plurality of recessed cavities in said textured surface portion; and then
(b) depositing a plurality of live nonembryonic progenitor cells on said textured surface portion in said recessed cavities so that said cells are protected from subsequent mechanical dislodgment therefrom; and then
(c) implanting said solid support in a subject.

38. The method according to claim 37, further comprising the step of:
(d) maintaining said substrate in said subject for a time sufficient for said cells to proliferate on said surface portion.

39. The method according to claim 38, further comprising the step of
(e) administering a test compound to said subject during said maintaining step.

40. The method according to claim 39, further comprising the step of:
(f) removing said solid support from said subject after said maintaining step.

41. The method according to claim 40, further comprising the step of:
(g) examining said cells to determine the effect of said test compound on said cells.

42. The method of claim 37, wherein said progenitor cells are selected from the group consisting of liver cells, pancreatic cells, intestinal cells, renal cells, and epithelial cells.
